(19) United States
(12) Patent Application Publication
(10) Pub. No.: US 2006/0211026 A1
(43) Pub. Date: Sep. 21, 2006

(54) Methods for assessing emphysema

(75) Inventors: Paula N. Belloni, Half Moon Bay, CA (US); Amy Berson, Palo Alto, CA (US); Irene Bailey-Healy, Cupertino, CA (US); Lada Markovtsova, Foster City, CA (US)

Correspondence Address: ROCHE PALO ALTO LLC;PATENT LAW DEPT. M/S A2-250, 3431 HILLVIEW AVENUE, PALO ALTO, CA, 94304, US

(73) Assignee: Roche Palo Alto LLC

(21) Appl. No.: 11/375,289

(22) Filed: Mar. 13, 2006

Related U.S. Application Data
(60) Provisional application No. 60/681,883, filed on May 16, 2005.
(60) Provisional application No. 60/662,677, filed on Mar. 17, 2005.

(51) Int. Cl. C12Q 1/68

(52) U.S. Cl. 435/6

(57) ABSTRACT

Emphysema and COPD are diagnosed, and the efficacy of therapeutic drug candidates for the treatment of emphysema and/or COPD is evaluated, by determining biomarkers selected from the group SpB, desmosine, VEGF, IGFBP2, MMP12, TIMP1, MMP9, Crabp2, Rbp1, Cyp26a1, Tgm2, Timp3, Adam17, Serpina1, Slpi, Col1a1, Eln, TGFβ1, TGFβ-RII, Sftpa1, Csf2, Cxcl1, Cxcl2, Cxcl5, IL-8Rβ, IL-8Rα, IL-6, TNF, EGF-R, Areg, PDGFα, HpGF, FGF7, Kdr, flt1, Angpt1, Tek, HIF1α, Hyou1, PGF, and tropoelastin.

### PRIORITY

This application claims priority from U.S. Ser. No. 60/681,883 filed May 16, 2005, and U.S. Ser. No. 60/662,677 filed Mar. 17, 2005, both incorporated herein by reference.

### FIELD OF THE INVENTION

This invention relates generally to methods of diagnosis, biomarkers, and screening techniques. More particularly, the invention relates to methods for assessing the severity and/or progression or regression of emphysema and chronic obstructive pulmonary disease (COPD), and methods for determining the efficacy of drugs that may be capable of treating said diseases.

### BACKGROUND OF THE INVENTION

Emphysema is defined as a loss of peripheral alveolar structure leading to reduced elastic recoil and subsequent decline in FEV₁. There is an urgent need to develop surrogate markers that may predict early onset of emphysema, and the usefulness of new candidate medicines. Recent preclinical studies suggest that retinoids, a class of compounds structurally related to vitamin A, may prevent the continued destruction and promote the repair and/or re-alveolarization of parenchymal lesions associated with emphysema.

Chronic Obstructive Pulmonary Disease (“COPD”) refers to a large group of lung diseases which prevent normal respiration. Approximately 11% of the population of the United States has COPD and available data suggests that the incidence of COPD is increasing. Currently, COPD is the fourth leading cause of mortality in the United States. COPD is a disease in which the lungs are obstructed due to the presence of at least one disease selected from asthma, emphysema and chronic bronchitis. The term COPD was introduced because these conditions often co-exist and in individual cases it may be difficult to ascertain which disease is responsible for causing the lung obstruction (1987 Merck Manual). Clinically, COPD is diagnosed by reduced expiratory flow from the lungs that is constant over several months and in the case of chronic bronchitis persists for two or more consecutive years. The most severe manifestations of COPD typically include symptoms characteristic of emphysema.

Emphysema is a disease where the gas-exchange structures (e.g., alveoli) of the lung are destroyed, which causes inadequate oxygenation that may lead to disability and death. Anatomically, emphysema is defined by permanent airspace enlargement distal to terminal bronchioles, which is characterized by reduced lung elasticity, decreased alveolar surface area and gas exchange and alveolar destruction that results in decreased respiration. Thus, the characteristic physiological abnormalities of emphysema are reduced gas exchange and expiratory gas flow.

Cigarette smoking is the most common cause of emphysema, although other environmental toxins may also contribute to alveoli destruction. The toxic compounds present in smoke can activate destructive processes that include the release of excessive amounts of proteases that overwhelm normal protective mechanisms, such as protease inhibitors present in the lung. The imbalance between proteases and protease inhibitors present in the lung may lead to elastin matrix destruction, elastic recoil loss, tissue damage, and continuous lung function decline. The rate of lung damage may be decreased by reducing the amounts of toxins in the lung (e.g., by ceasing to smoke). However, the damaged alveolar structures are not repaired and lung function is not regained. At least four different types of emphysema have been described according to their locations in the secondary lobule: panlobar emphysema, centrilobular emphysema, distal lobular emphysema and paracicatrical emphysema.

The major symptom of emphysema is chronic shortness of breath. Other important symptoms of emphysema include chronic cough, coloration of the skin caused by lack of oxygen, shortness of breath after minimal physical activity, and wheezing. Additional symptoms that may be associated with emphysema include vision abnormalities, dizziness, temporary cessation of respiration, anxiety, swelling, fatigue, insomnia and memory loss. Emphysema is typically diagnosed by a physical examination that shows decreased and abnormal breathing sounds, wheezing and prolonged exhalation. Pulmonary function tests, reduced oxygen levels in the blood and a chest X-ray may be used to confirm a diagnosis of emphysema.

A need exists for compounds useful in the treatment of emphysema and COPD. However, there are few suitable clinical indicators of drug efficacy. Currently, the accepted indicator is reduction in the rate of decline in forced expiratory volume in one second (FEV₁). Because FEV₁ declines normally with age (and at an accelerated pace in patients with emphysema and/or COPD), and has a high degree of variability, clinical trials capable of demonstrating a statistically significant improvement require enrollment of many patients and/or time courses of 1 to 3 years (see, e.g., M-L Wang et al., J Occup Environ Med (2004) 46(6):591-95; P. Llewellin et al., Respirol (2002) 7:333-37; J. Zhang et al., Eur Respir J (2002) 20:1102-09). This inhibits the introduction of new drugs, due to the high cost of enrolling large patient populations, and the lengthy delays associated with long trials. Thus, the need for new drugs may be addressed in part by providing an improved clinical measure of efficacy.

Biomarkers are characteristics that may be objectively measured and evaluated as an indicator of normal biological processes, pathogenic processes, or pharmacologic responses to a therapeutic intervention (Biomarkers Definitions Working Group, Clin Pharmacol Ther (2001) 69(3):89-95). In the clinical setting, biomarkers are observable features or detectable substances that correlate well with a disease state or therapeutic outcome. T. Betsuyaku et al., Am J Respir Crit Care Med (2003) 168:222-27 studied concentrations of extracellular matrix metalloproteinase inducer (“EMMPRIN”) and interstitial collagenase (aka matrix metalloproteinase 1, or “MMP1”) in bronchoalveolar lavage fluid (“BAL”) obtained from smokers with and without emphysema, former smokers with and without emphysema, and control subjects who had never smoked. Both EMMPRIN and MMP1 were elevated in BAL obtained from smokers: however, there was no correlation with emphysema disease status.

I. S. Patel et al., Am J Respir Crit Care Med (2004) 170:400-07 studied levels of IL-6 and IL-8, and the presence of bacteria in sputum in COPD patients, and assessed emphysema and bronchiectasis (pathological destruction and dilation of the larger airways) by high resolution computed tomography (HRCT). The authors found that sputum IL-8 and IL-6 levels were higher patients with significant bronchiectasis than in patients with lower bronchiectasis scores. However, there was apparently no correlation with emphysema scores.

G. Turato et al., Am J Respir Crit Care Med (2002) 166:105-10 found that patients with severe COPD exhibit an enhanced inflammatory response in the small airways, based on CD45⁺ cell counts in the airway walls. However, the method involved histological examination of resected lung tissue, which is problematic for routine diagnostic or clinical trial use.

A. Ekberg-Jansson et al., Respir Med (2001) 95:363-73 measured human neutrophil lipocalin (HNL), Clara cell protein 16 (“CC-16”), interleukin-8 (IL-8), lysozyme, myeloperoxidase (MPO), interleukin-1β (IL-1β) in serum, BAL, and bronchial lavage (BL) (except that IL-1β was not determined in serum). The authors compared smokers with and without emphysematous lesions, and found elevated HNL and MPO, and depressed CC-16 in blood, and increased HNL (but not MPO or CC-16) in BAL. There were no significant differences in IL-8, IL-1β, or lysozyme.

F. Cocci et al., Int J Biochem Cell Biol (2002) 34:594-604 reported that urinary concentrations of desmosine correlate with emphysema.

### SUMMARY OF THE INVENTION

The invention provides methods for diagnosing COPD and emphysema, and for monitoring clinical progress in the treatment of COPD and/or emphysema. The invention also provides methods for determining the efficacy of a drug candidate for the treatment of COPD and/or emphysema.

One aspect of the invention is a method for diagnosing COPD or emphysema by determining the relative level of a biomarker in said biological sample, wherein said biomarker is selected from the group consisting of lung specific surfactant protein B (SpB), desmosine, VEGF, IGFBP2, MMP12, TIMP1, MMP9, Crabp2, Rbp1, Cyp26a1, Tgm2, Timp3, Adam17, Serpina1, Slpi, Col1a1, TGFβ1, TGFβ-RII, Sftpa1, Csf2, Cxcl1, Cxcl2, Cxcl5, IL-8Rβ, IL-8Rα, IL-6, TNF, EGF-R, Areg, PDGFα, HpGF, FGF7, Kdr, flt1, Angpt1, Tek, HIF1α, Hyou1, PGF, and tropoelastin; and determining whether said relative level is higher or lower than the expected level of said biomarker. The biological sample is preferably obtained from a subject.

Another aspect of the invention is method for measuring the effect of a drug candidate for the treatment of COPD or emphysema on a subject by determining the baseline level of a biomarker in a first biological sample, wherein said biomarker is selected from the group consisting of SpB, desmosine, VEGF, IGFBP2, MMP12, TIMP1, MMP9, Crabp2, Rbp1, Cyp26a1, Tgm2, Timp3, Adam17, Serpina1, Slpi, Col1al1, Eln, TGFβ1, TGFβ-RII, Sftpa1, Sftpb, Csf2, Cxcl1, Cxcl2, Cxcl5, IL-8Rβ, IL-8Rα, IL-6, TNF, EGF-R, Areg, PDGFα, HpGF, FGF7, Kdr, flt1, Angpt1, Tek, HIF1α, Hyou1, PGF, and tropoelastin (protein); determining the level of said biomarker in a second biological sample; and determining whether the level of said biomarker in said second sample is higher or lower than the baseline level of said biomarker; the biological samples being obtained from a subject before and after administration of a drug candidate.

### BRIEF DESCRIPTION OF THE DRAWING

FIG. 1 depicts the amount of tropoelastin (in ng) measured in rats after treatment as set forth in Example 1(B). N=rats not receiving smoke; Smk=rats smoked for 8 months, but untreated; Veh=rats receiving only vehicle for 30 days after smoking 8 months; ATRA=rats receiving 3 mg/kg all-trans retinoic acid for 30 days after smoking 8 months; 0.07=rats receiving 0.07 mg/kg R667 for 30 days after smoking 8 months; 0.15=rats receiving 0.15 mg/kg R667 for 30 days after smoking 8 months; 0.7=rats receiving 0.7 mg/kg R667 for 30 days after smoking 8 months.

### DETAILED DESCRIPTION OF THE INVENTION

Definitions

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

“Agonist” refers to a compound that enhances the activity of another compound or receptor site.

“Antagonist” refers to a compound that diminishes or prevents the action of another compound or receptor site.

The term “biomarker” refers to an objectively measurable quantity or characteristic that correlates with a normal biological process, a pathological (disease) state or process, or a pharmacological response to a drug candidate. A “clinical endpoint” refers to a characteristic or variable that reflects how a patient feels, functions, or survives. A “surrogate endpoint” refers to a biomarker that is intended to substitute for a clinical endpoint. Biomarkers of the invention correlate with severity of COPD and emphysema, and with the treatment of COPD and emphysema.

The term “biological sample” refers to a portion of blood, serum, BAL, bronchial lavage, bronchial or bronchiolar brushing, tissue, phlegm, urine, saliva, and the like obtained from a subject, from which a biomarker may be determined. The term “BAL” refers to bronchoalveolar lavage, which is typically obtained by instilling and removing a quantity of fluid (such as buffered saline) in a portion of the lung.

The term “drug candidate” refers to a compound or preparation which is to be tested for possible effect in the treatment of a disease state in an animal, regardless of whether said drug candidate has any known biological activity.

The term “ATRA” refers to all-trans retinoic acid.

The term “R667” refers to the compound 4-[2-(5,5,8,8-tetramethyl-3-pyrazol-1-ylmethyl-5,6,7,8-tetrahydronaphthalen2-yl)-vinyl]-benzoic acid, 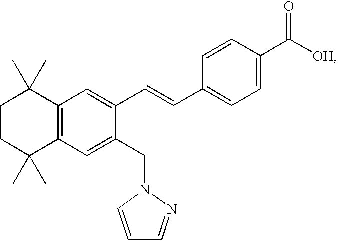
which is further described in U.S. Pat. No. 6,777,418.

The term “relative level” refers to a measure of the concentration, activity, expression level, or amount of a biomarker present. A relative level may be determined quantitatively, for example by measuring the concentration or mass of the biomarker present in a sample, or for example by determining the quantity of mRNA encoding the biomarker that is expressed in a relevant cell population or tissue (e.g., in macrophages, neutrophils, eosinophils, basophils, and the like). Alternatively, the relative level may be determined more qualitatively, e.g., as being above or below a set threshold level. The threshold level may correspond to an average or median level of the biomarker in healthy subjects, or may correspond to the level at which a diagnosis of disease is made (for example, the threshold level may correspond to the lowest level that correlates with a diagnosis of COPD).

The term “expected level” refers to the level outside of which a diagnosis of disease is made, whether disease is indicated by levels in excess of the expected level or below the expected level. A level of expression within the expected level corresponds to a “normal” or non-disease state. For example, if the relative expression level of a selected gene is 4.2±1.7 within a typical population or sample, a measured relative expression level of less than 2.5 or greater than 5.9 corresponds to a disease state.

“Desmosine” refers to the compound 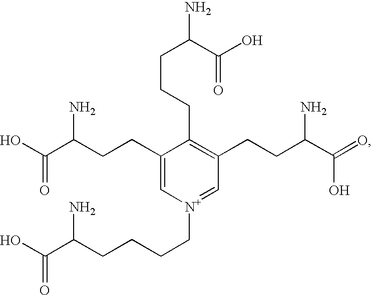
a normal constituent of elastin formed by the enzymatic condensation of four Lys residues, thereby cross-linking chains of elastin.

“IGFBP2” refers to human insulin-like growth factor binding protein-2, and homologous proteins expressed in the lung (including by myeloid cells) in other subject species.

“MMP1” refers to human collagenase 1 and homologous proteins expressed in the lung (including by myeloid cells) in other subject species.

“MMP8” refers to human collagenase 2 and homologous proteins expressed in the lung (including by myeloid cells) in other subject species.

“MMP9” refers to human gelatinase B and homologous proteins expressed in the lung (including by myeloid cells) in other subject species.

“MMP12” refers to human macrophage metalloelastase and homologous proteins expressed in the lung (including by myeloid cells) in other subject species.

“SpB” and “SFTPB” refers to human lung surfactant protein B (also called lung-specific surfactant protein B), and homologous proteins expressed in the lung in other subject species.

“RAR” refers to the human retinoic acid receptor, and homologous proteins expressed in other subject species. RAR in humans is known to exist in three different subtypes: RARα, RARβ, and RARγ.

“VEGF” and “VEGFa” refers to human vascular endothelial growth factor, and homologous proteins expressed in the lung (including by myeloid cells) in other subject species. “VEGF₁₆₅” refers specifically to the 165 amino acid VEGF isoform, and homologous proteins in other species.

“TIMP1” refers to human tissue inhibitor of metalloprotease-1, and homologous proteins expressed in the lung (including by myeloid cells) in other subject species.

“Timp3” refers to tissue inhibitor of metalloprotease-3 (Sorsby fundus dystrophy, pseudoinflammatory), and homologous proteins expressed in the lung (including by myeloid cells) in other subject species.

“Tropoelastin” and “ELN” refers to human tropoelastin, and homologous proteins expressed in the lung (including by myeloid cells) in other subject species.

“Crabp2” refers to cellular retinoic acid binding protein 2, and homologous proteins expressed in the lung (including by myeloid cells) in other subject species.

“Rbp1” refers to cellular retinol binding protein 1, and homologous proteins expressed in the lung (including by myeloid cells) in other subject species.

“Cyp26a1” refers to cytochrome p450, family 26, subfamily A, polypeptide 1, and homologous proteins expressed in the lung (including by myeloid cells) in other subject species.

“Tgm2” refers to tissue-type transglutaminase, and homologous proteins expressed in the lung (including by myeloid cells) in other subject species.

“Adam17” refers to A distinegrin and metalloproteinase domain 17 (TNFα, converting enzyme), and homologous proteins expressed in the lung (including by myeloid cells) in other subject species.

“Serpina1” refers to serine (or cysteine) proteinase inhibitor, clade A (α1 anti-proteinase, antitrypsin), member 1, and homologous proteins expressed in the lung (including by myeloid cells) in other subject species.

“Slpi” refers to secretory leukocyte protease inhibitor, and homologous proteins expressed in the lung (including by myeloid cells) in other subject species.

“Col1a1” refers to collagen type 1, α1, and homologous proteins expressed in the lung (including by myeloid cells) in other subject species.

“Tgfb1” and “TGFβ1” refers to transforming growth factor-β1, and homologous proteins expressed in the lung (including by myeloid cells) in other subject species.

“Tgfbr2” and TGFβ-RII” refers to transforming growth factor β receptor II, and homologous proteins expressed in the lung (including by myeloid cells) in other subject species.

“Sftpa1” refers to pulmonary-associated surfactant protein A1, and homologous proteins expressed in the lung (including by myeloid cells) in other subject species.

“Csf2” and “CSF2” refer to colony stimulating factor II (granulocyte-macrophage), and homologous proteins expressed in the lung (including by myeloid cells) in other subject species.

“Cxcl1” refers to chemokine (C-X-C motif) ligand 1 (Gro1), and homologous proteins expressed in the lung (including by myeloid cells) in other subject species.

“Cxcl2” refers to chemokine (C-X-C motif) ligand 2 (MIP2), and homologous proteins expressed in the lung (including by myeloid cells) in other subject species.

“Cxcl5” and “ENA78” refers to chemokine LIX, and homologous proteins expressed in the lung (including by myeloid cells) in other subject species.

“Il8rb/CXCR2” and “IL-8Rβ” refers to interleukin 8 receptor β, and homologous proteins expressed in the lung (including by myeloid cells) in other subject species.

“Il8ra/CXCR1” and “IL-8Rα” refers to interleukin 8 receptor α, and homologous proteins expressed in the lung (including by myeloid cells) in other subject species.

“Il6” refers to interleukin-6, and homologous proteins expressed in the lung (including by myeloid cells) in other subject species.

“EGFR” refers to epidermal growth factor receptor, and homologous proteins expressed in the lung (including by myeloid cells) in other subject species.

“Areg” refers to amphiregulin, and homologous proteins expressed in the lung (including by myeloid cells) in other subject species.

“PDGFA” and “PDGFα” refer to platelet derived growth factor α, and homologous proteins expressed in the lung (including by myeloid cells) in other subject species.

“Hgf” and “HpGF” refer to hepatocyte growth factor, and homologous proteins expressed in the lung (including by myeloid cells) in other subject species.

“Kdr” refers to kinase insert domain protein receptor, and homologous proteins expressed in the lung (including by myeloid cells) in other subject species.

“Flt1” refers to FMS-like tyrosine kinase 1, and homologous proteins expressed in the lung (including by myeloid cells) in other subject species.

“Angpt1” refers to angiopoietin-1, and homologous proteins expressed in the lung (including by myeloid cells) in other subject species.

“Tek” refers to endothelial-specific receptor tyrosine kinase, and homologous proteins expressed in the lung (including by myeloid cells) in other subject species.

“Hif1a” and “HIF-1α” refers to hypoxia inducible factor 1, α subunit, and homologous proteins expressed in the lung (including by myeloid cells) in other subject species.

“Hyou1” refers to hypoxia up-regulated 1, and homologous proteins expressed in the lung (including by myeloid cells) in other subject species.

“Pgf” refers to placental growth factor, and homologous proteins expressed in the lung (including by myeloid cells) in other subject species.

The term “homologous” as used herein refers to a protein that performs substantially the same function in another subject species and shares substantial sequence identity, to the extent that they are recognized in the art as being different versions of the same protein, differing primarily in the species in which they are found. Thus, for example, human MMP9, mouse MMP9, and rat MMP9 are all considered homologous to each other.

“Modulator” means a molecule that interacts with a target. The interactions include, but are not limited to, agonist, antagonist, and the like, as defined herein.

“Disease” and “Disease state” means any disease, condition, symptom, disorder or indication.

“Subject” includes mammals and birds. “Mammals” means any member of the mammalia class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. The term “subject” does not denote a particular age or sex.

“Treating” or “treatment” of a disease state includes (i) preventing the disease state, i.e. causing the clinical symptoms of the disease state not to develop in a subject that may be exposed to or predisposed to the disease state, but does not yet experience or display symptoms of the disease state; (ii) inhibiting the disease state, i.e., arresting the development of the disease state or its clinical symptoms; or (iii) relieving the disease state, i.e., causing temporary or permanent regression of the disease state or its clinical symptoms.

General Method

The invention provides methods for diagnosing the presence and severity of COPD and emphysema, and for determining the reduction of COPD and emphysema by a drug candidate.

In order to accelerate the process of activity and efficacy determination regarding the treatment of emphysema and/or COPD, we have determined that a number of biomarkers correlate with severity of disease and its therapeutic treatment. These biomarkers are determined more readily than the accepted endoint, ΔFEV₁ (change in FEV₁, or more specifically, the reduction in the rate of decline in FEV₁), which must usually be measured over a period of one to three years in order to obtain a statistically significant result. Biomarkers of the invention may be determined after much shorter periods. Preferably, one or more biomarkers are determined at least about 8 hours after administration of a drug candidate to a subject; more preferably, one or more biomarkers are determined at least about 24 hours after administration of a drug candidate; still more preferably, one or more biomarkers are determined at least about 7 days after administration of a drug candidate; still more preferably, one or more biomarkers are determined at least about 14 days after administration of a drug candidate; still more preferably, one or more biomarkers are determined at least about 30 days after administration of a drug candidate. Biomarkers are preferably determined within about six months of administration of a drug candidate to a subject; more preferably within about four months; more preferably within about three months; most preferably at multiple time points between one day and six months from administration of the drug candidate. A baseline level for each biomarker is preferably measured prior to administration of any drug candidate or control.

The number and selection of biomarkers will depend upon the biological samples available, and the methods available for quantifying the biomarkers. In general, it is preferred to use more than one biomarker; preferably at least two biomarkers; more preferably at least three biomarkers.

The relative level of a biomarker can be determined based on protein or mRNA, in the case of proteins, or by concentration or amount (e.g., in the case of desmosine and ELN), depending on the biological sample. Compounds such as desmosine can be determined by standard chemical methods, including for example immunoassay methods (see, e.g., F. Cocci et al., Int J Biochem Cell Biol (2002) 34:594-604) and spectroscopic methods (e.g., NMR, IR, HPLC, capillary zone electrophoresis, GC-MS, and the like): see, e.g., S. Ma et al., Proc Natl Acad Sci USA (2003) 100(22):12941-43. Desmosine can be determined in urine, BAL, serum, and other biological samples.

Protein biomarkers such as SFTPB, VEGF, IGFBP2, MMP9, MMP12, TIMP1, and ELN can be measured directly by physical/chemical techniques such as gel electrophoresis, HPLC, mass spectroscopy, and proteomic techniques; immunoassay techniques, such as ELISA, competitive assays, sandwich assays, and the like; and by assays of biological activity (for example, as a ligand and/or an enzyme), by measuring that activity in the biological sample by methods known in the art, through two-hybrid assay systems, and the like. Commercial assays are available for many or most of the above-mentioned biomarkers, or are described in the art. Suitable biological samples include BAL, bronchial or bronchiolar brushing, sputum, blood, serum, urine, lung tissue, and the like. One can determine biomarkers from a variety of different sample sources, for example, one can determined IGFBP2 in serum while determining SFTPB in BAL. 1791 Alternatively, one can determine protein biomarkers indirectly by determining the change in mRNA transcription in relevant cells. (Or, equivalently, one can determine mRNA biomarkers that correspond to the above-mentioned protein biomarkers.) The determination of mRNA transcription level can be performed using any suitable quantitative or semi-quantitative method, including without limitation Northern blot; microarray techniques; RT-PCR and other quantitative and semi-quantitative DNA and mRNA amplification methods; and the like. For example, to perform RT-PCR, one can extract total RNA from the biological sample, treat it with Dnase1 and convert it to cDNA using a reverse transcriptase such as Multiscribe reverse transcriptase (Applied Biosystems Inc., Foster City, Calif.). A cDNA “SYBR green” real-time quantitative PCR assay can then be performed using the cDNA as template, and analyzed using an ABI PRISM 7900 Sequence Detector.

### UTILITY/INDUSTRIAL APPLICABILITY

Methods of the invention are useful for diagnosing emphysema and/or COPD in patients, for following the progress of clinical trials regarding emphysema and/or COPD therapeutics, and for determining the biological activity of drug candidates designed to treat emphysema and/or COPD.

### EXAMPLES

The following preparations and examples are given to enable those skilled in the art to more clearly understand and to practice the present invention. They should not be considered as limiting the scope of the invention, but merely as being illustrative and representative thereof.

### Example 1

### Biomarkers in Rats

(A) Rats were exposed to cigarette smoke (10 cigarettes/day) for 8 months, following a smoking protocol similar to that of A. F. Ofulue et al., AJP Lung Cell Mol Physiol (1999) 277:97-105. Following the 6 month exposure, 14 rats were sacrificed, and samples of BAL, plasma and lungs obtained and frozen, along with samples from 17 naïve rats. Rats in other arms of the study were administered doses of drug candidates or vehicle for 30 days. After 30 days, BAL, plasma, and lungs were collected from the remaining animals.

BAL was examined for desmosine, SpB, and MMP9. Lungs were subjected to histological analysis, and mRNA expression analysis by RT-PCR. Untreated smoking rats exhibited elevated desmosine and MMP9 levels in BAL, upregulated expression of MMP9, IL-6, IL-8 mRNA, and down-regulated expression of IGFBP2, SpB, SpA, and VEGF. Treatment with active drug candidates decreased elastin fragments in BAL (92%), decreased MMP9 and MMP12 levels (50%), decreased inflammatory cells in BAL (60%), and increased mature elastin in lung tissue. (80%).

(B) Adult male Sprague-Dawley rats were exposed daily to 10 cigarettes/rat for 8 months (control rats were exposed to air) to establish emphysema lesions. Thereafter, the rats were treated orally with ATRA (3 mg/kg), R667, or vehicle daily for 30 days. Supernatant from homogenized lung tissue was analyzed for concentration of tropoelastin by ELISA (using rabbit anti-tropoelastin; Elastin Products Co.). The results are shown in FIG. 1. The data shows that tropoelastin is depressed by smoking, and is partially restored by treatment with ATRA or R667.

RNA prepared from snap-frozen lung right lobes was analyzed for gene expression by quantitative PCR, using ABI Taqman following standard procedures. The results were normalized to 18s RNA expression, and calculated relative to the expression levels of the 8 months smoked rats, and are shown in Table 1 below.

TABLE 1
Gene expression relative to 8 months smoked
rats (untreated) by quantitative PCR

| | 8 months | | R667 (mg/kg) | | | | Ve- |
|---|---|---|---|---|---|---|---|
| Gene | Control | Smoke | 0.07 | 0.15 | 0.7 | ATRA | hicle |
| Crabp2 | 2.1 | 1.0 | 2.0 | 1.5 | 1.4 | 1.7 | 1.9 |
| Rbp1 | 5.0 | 1.0 | 2.1 | 3.5 | 3.9 | 3.7 | 2.2 |
| Cyp26a1 | 3.3 | 1.0 | 2.2 | 3.9 | 3.7 | 252.7 | 1.6 |
| Tgm2 | 14.8 | 1.0 | 16.2 | 28.3 | 24.6 | 59.1 | 29.5 |
| Mmp9 | 1.2 | 1.0 | 1.7 | 1.5 | 1.9 | 27.3 | 1.3 |
| Mmp12 | 0.4 | 1.0 | 4.9 | 5.9 | 7.3 | 1.7 | 2.3 |
| Timp3 | 8.8 | 1.0 | 3.5 | 2.9 | 4.8 | 0.6 | 1.2 |
| Adam17 | 7.1 | 1.0 | 3.5 | 3.6 | 7.8 | 2.8 | 2.3 |
| Serpina1 | 4.6 | 1.0 | 2.3 | 1.5 | 2.9 | 1.4 | 1.4 |
| Slpi | 1.6 | 1.0 | 3.6 | 1.6 | 4.4 | 4.9 | 1.8 |
| Colla1 | 28.3 | 1.0 | 7.1 | 8.4 | 7.9 | 14.9 | 9.3 |
| Eln | 25.1 | 1.0 | 5.6 | 6.1 | 9.4 | 10.5 | 4.6 |
| Tgfb1 | 2.1 | 1.0 | 1.4 | 2.0 | 3.2 | 1.6 | 1.6 |
| Tgfbr2 | 2.2 | 1.0 | 2.0 | 1.5 | 2.1 | 0.9 | 1.0 |
| Sftpa1 | 4.1 | 1.0 | 5.4 | 4.4 | 7.0 | 12.6 | 8.0 |
| Sftpb | 1.6 | 1.0 | 1.9 | 2.1 | 2.9 | 1.5 | 1.9 |
| Csf2 | 3.8 | 1.0 | 1.5 | 1.9 | 2.9 | 0.9 | 1.0 |
| Cxcl1 | 1.1 | 1.0 | 3.3 | 2.9 | 6.5 | 2.2 | 1.8 |
| Cxcl2 | 0.8 | 1.0 | 2.3 | 1.6 | 3.4 | 0.8 | 0.7 |
| Cxcl5 | 1.4 | 1.0 | 50.1 | 50.0 | 81.6 | 43.0 | 41.7 |
| Il8rb/CXCR2 | 1.2 | 1.0 | 2.0 | 2.0 | 2.5 | 0.5 | 1.0 |
| Il8ra/CXCR1 | 3.6 | 1.0 | 3.8 | 4.5 | 4.2 | 5.7 | 2.3 |
| Il6 | 1.1 | 1.0 | 1.8 | 2.4 | 4.5 | 2.7 | 2.5 |
| TNF | 1.4 | 1.0 | 3.3 | 4.2 | 4.0 | 3.1 | 2.0 |
| EGFR | 6.5 | 1.0 | 4.8 | 3.2 | 4.0 | 2.1 | 1.7 |
| Areg | 3.4 | 1.0 | 5.9 | 3.7 | 7.2 | 2.4 | 3.4 |
| PDGFA | 6.0 | 1.0 | 4.3 | 2.9 | 4.5 | 1.6 | 2.6 |
| HpGF | 2.4 | 1.0 | 3.0 | 3.1 | 5.6 | 2.0 | 1.9 |
| FGF7 | 3.1 | 1.0 | 2.1 | 2.1 | 1.6 | 1.2 | 1.7 |
| VEGFa | 5.5 | 1.0 | 1.6 | 2.3 | 2.9 | 1.3 | 1.2 |
| Kdr | 4.4 | 1.0 | 1.3 | 1.9 | 1.7 | 0.7 | 1.3 |
| Flt1 | 3.8 | 1.0 | 1.7 | 1.8 | 2.6 | 0.8 | 0.8 |
| Angpt1 | 4.5 | 1.0 | 3.8 | 1.8 | 3.1 | 0.8 | 0.9 |
| Tek | 2.2 | 1.0 | 1.4 | 1.3 | 1.3 | ND | 1.2 |
| Hif1a | 2.1 | 1.0 | 1.7 | 1.4 | 2.0 | 1.3 | 1.2 |
| Hyou1 | 4.8 | 1.0 | 2.7 | 3.3 | 5.8 | 2.4 | 1.9 |
| Pgf | 3.1 | 1.0 | 1.6 | 0.9 | 2.7 | 0.5 | 0.5 |

Three individual lung right lobes from each condition were processed to extract mRNA according to standard Affymetrix protocols, and samples hybridized to Affymetrix Rat Expression Set 230 microarrays. Gene expression was analyzed using the Bioconductor package (Gentleman et al., Genome Biol (2004) v. 5), and differentially expressed genes selected using ANOVA model followed by Dunnett's test. This data, calculated as fold expression relative to the 8 months smoked rat, is shown in Table 2 below.

TABLE 2
Expression levels from microarray

| | ANOVA | | | R667 (mg/kg) | | |
|---|---|---|---|---|---|---|
| Gene | p value | Smoke | Vehicle | 0.07 | 0.15 | 0.7 |
| ELN | 1.2 × 10⁻⁴ | 1.0 | 3.6 | 2.8 | 3.1 | 3.8 |
| VEGF | 1.3 × 10⁻² | 1.0 | 1.7 | 1.7 | 1.5 | 2.4 |

The results demonstrate that smoking suppresses the expression of a number of genes, including genes related to retinoid activity (Crabp2, Rbp1, Cyp26a1, Tgm2), protease or anti-protease activity (MMP9, MMP12, Timp3, Adam17, Serpina1, Slpi), extracellular matrix and alveolar proteins (Col1a1, Eln, TGFβ1, TGFβ-RII, Sftpa1, Sftpb, Csf2), inflammation and tissue injury (Cxcl1, Cxcl2, Cxcl5, IL-8Rβ, IL-8Rα, IL-6, TNF), growth factors (EGF-R, Areg, PDGFα, HpGF, FGF7), and angiogenesis factor and receptors (VEGFa, Kdr, flt1, Angpt1, Tek, HIF1α, Hyou1, PGF). The results further demonstrate that R667 was able to restore expression of these genes to near-normal levels. This restoration correlated with restoration of tropoelastin expression in the lungs, an indication of tissue regrowth and repair.

### Example 2

### Biomarkers in Humans

A randomized, multi-center, double blind, parallel-group study was designed to asses the safety of daily doses of a drug candidate (vs. placebo) in humans with moderate to severe emphysema, over four weeks. A total of 86 patients were enrolled, of which 24 were also randomized to examination of biomarkers. All non-control patients were required, inter alia, to be 50 years of age or older, have symptomatic emphysema at stable clinical condition, exhibit physiologic evidence of moderate to severe emphysema (DL_{CO}<50% of predicted value adjusted for gender, age, height, and hemoglobin; and FEV₁≦60% predicted after bronchodilator administration, adjusted for gender, age, height, and hemoglobin), exhibit breathlessness of at least 1 on the Modified Medical Research Council Scale, and have radiologic confirmation of emphysema upon visual examination of a chest CT scan. Patients were excluded for, inter alia, depression, psychiatric disorders requiring medication or hospitalization, solitary nodules in the lung requiring further medical intervention, maintenance therapy with oral steroids, giant bullous disease, hypertriglyceridemia ≧300 mg/dL, unexplained weight loss of ≧10% total body weight over the previous 6 months, or body mass index <19 Kg/m², or history of sensitivity to retinoids.

Plasma samples were collected prior to drug administration, during the 2^{nd} week after administration (20-24 hours after dosing), and during the 4^{th} week after administration (20-24 hours after dosing). BAL and peripheral bronchiolar brushings were obtained prior to treatment, and four weeks after initial dosing (within 25-31 days of the first dose). Patients contributing BAL and peripheral bronchiolar brushings were further examined by bronchoscopy, if they exhibited post bronchodilator FEV1≧40% of predicted at enrollment; O₂ saturation on room air ≧90% by pulse oximetry; Pa_{CO2}<45 mmHg (by arterial blood gas); and absence of coagulopathy (platelet count >100,000/mm³, and PT and PTT<1.2× the upper limit of normal).

Plasma samples were analyzed by immunoassay (using commercially available kits) for the concentrations of MMP1, MMP9, MMP12, TIMP1 (Amersham RPN-2611), TIMP2, FGF7, VEGF (Amersham RPN2279), IGFBP2 (R&D Systems MAB6741, 674-B2, BAF674) and HGF. BAL samples were analyzed for MMP1, MMP9, MMP12, TIMP1, TIMP2, FGF7, VEGF, IGFBP2, HGF, desmosines (desmosine and isodesmosine), elastin fragments, and surfactant proteins A and B. Bronchiolar brushings were analyzed for MMP1, MMP9, MMP12, TIMP1, TIMP2, FGF7, VEGF, IGFBP2, HGF, surfactant proteins A and B, RARα, RARβ, RARγ, and p21. Bronchiolar brushings were analyzed for mRNA expression of MMP1, MMP9, MMP12, TIMP1, TIMP2, FGF7, VEGF, IGFBP2, HGF, surfactant proteins A and B, RARα, RARβ, RARγ, and p21 by RT-PCR using TaqMan gene expression assays.

SpB in BAL was found to be highest in normal patients who had never smoked (average 51.4 ng/mg), lower in smokers asymptomatic for emphysema (13.9 ng/mg), and lowest in symptomatic emphysema patients (8.5 ng/mg). SpB in BAL also correlated (ρ=0.59, p=0.007) with FEV₁ at baseline, and increased with increasing doses of the drug candidate. After four weeks, SpB in BAL increased in accordance with the dose of the drug candidate.

Desmosine, which is released when elastin is degraded, was found highest in the BAL of emphysema patients at baseline (37 pmol/mg protein), lower in asymptomatic smokers (12 pmol/mg protein), and lowest in non-smokers (8 pmol/mg protein). BAL desmosine was reduced in patients receiving the drug candidate after four weeks.

VEGF in BAL was highest in control patients (120 pg/ml), lower in asymptomatic smokers (34 pg/ml), and lowest in emphysema patients (20 pg/ml). VEGF concentrations in BAL increased after four weeks of treatment with the drug candidate.

IGFBP2 in plasma correlated with age and body mass index at baseline, and was negatively correlated with DL_{CO}. IGFBP2 in plasma was elevated in emphysema patients (614 ng/ml) and smokers (501 ng/ml) compared to controls (302 ng/ml) and healthy donors (115 ng/ml). Plasma IGFBP2 levels decreased after four weeks of treatment with the drug candidate.

The results demonstrate that SpB, desmosine, VEGF, and IGFBP2 are effective biomarkers in emphysema/COPD.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

1. A method for measuring COPD or emphysema in a subject, comprising:
obtaining a biological sample from said subject;
determining the relative level of a biomarker in said biological sample, wherein said biomarker is selected from the group consisting of lung specific surfactant protein B (SpB), desmosine, VEGF, IGFBP2, MMP12, TIMP1, MMP9, Crabp2, Rbp1, Cyp26a1, Tgm2, Timp3, Adam17, Serpina1, Slpi, Col1a1, Eln, TGFβ1, TGFβ-RII, Sftpa1, Csf2, Cxcl1, Cxcl2, Cxcl5, IL-8Rβ, IL-8Rα, IL-6, TNF, EGF-R, Areg, PDGFα, HpGF, FGF7, Kdr, flt1, Angpt1, Tek, HIF1α, Hyou1, PGF, and tropoelastin; and
determining whether said relative level is higher or lower than the expected level of said biomarker.

2. The method of claim 1, wherein said biological sample comprises bronchoalveolar lavage (BAL).

3. The method of claim 2, wherein said biomarker is selected from the group consisting of SpB, desmosine, VEGF, IGFBP2, TIMP1, MMP9, and tropoelastin.

4. The method of claim 3, wherein said biomarker comprises SpB and desmosine.

5. The method of claim 3, wherein said biomarker comprises SpB, IGFBP2, and VEGF.

6. The method of claim 1, wherein said biological sample comprises serum.

7. The method of claim 6, wherein said biomarker is selected from the group consisting of VEGF, IGFBP2, and desmosine.

8. The method of claim 1, wherein said biological sample comprises serum and BAL.

9. The method of claim 1, wherein said relative level is determined by quantitative mRNA PCR (RT-PCR).

10. A method for measuring the effect of a drug candidate for the treatment of COPD or emphysema on a subject, comprising:
obtaining a first biological sample from said subject;
determining the baseline level of a biomarker in said first biological sample, wherein said biomarker is selected from the group consisting of SpB, desmosine, VEGF, IGFBP2, MMP12, TIMP1, MMP9, and tropoelastin;
administering said drug candidate;
obtaining a second biological sample from said subject;
determining the level of said biomarker in said second biological sample; and
determining whether the level of said biomarker in said second sample is higher or lower than the baseline level of said biomarker in said first sample.

11. The method of claim 10, wherein said subject comprises a rat or mouse.

12. The method of claim 10, wherein said subject comprises a human.

13. The method of claim 10, wherein said biological sample is selected from the group consisting of blood, serum, urine, and BAL.

14. The method of claim 11, wherein said biological sample comprises lung tissue.

15. The method of claim 10, wherein said second biological sample is obtained at least six hours after administering said drug candidate.

16. The method of claim 10, wherein said second biological sample is obtained-no more than six months after administering said drug candidate.
